(19) United States
(12) Patent Application Publication
(10) Pub. No.: US 2011/0294682 A1
(43) Pub. Date: Dec. 1, 2011

(54) Polynucleotides Associated With Age-Related Macular Degeneration and Methods for Evaluating Patient Risks

(75) Inventors: Johanna M. Seddon, Boston, MA (US); Mark J. Daly, Arlington, MA (US)

(73) Assignees: THE GENERAL HOSPITAL CORPORATION, Boston, MA (US); TUFTS MEDICAL CENTER, INC., Boston, MA (US)

(21) Appl. No.: 12/989,771

(22) PCT Filed: Apr. 27, 2009
(86) PCT No.: PCT/US09/41778

§ 371 Date: Apr. 8, 2011

Related U.S. Application Data
(60) Provisional application No. 61/048,361, filed on Apr. 28, 2008.

(51) Int. Cl. C40B 30/04; C12Q 1/68

(52) U.S. Cl. 506/9; 435/6.11

(57) ABSTRACT

Disclosed are methods for diagnosing AMD or a susceptibility for AMD by identifying one or more markers associated with peripheral retinal phenotypes.

### GOVERNMENT SUPPORT

This invention was made with government support under EY011309 awarded by the National Institutes of Health. Additional funding was provided by the National Eye Institute (N01-EY-0-2127) and grant U54 RR020278 from the National Center for Research Resources. The government may have certain rights in the invention.

### BACKGROUND

Age-related macular degeneration (AMD) is the most common geriatric eye disorder leading to blindness. Macular degeneration is responsible for visual handicap in what is estimated conservatively to be approximately 16 million individuals worldwide. Among the elderly, the overall prevalence is estimated between 5.7% and 30% depending on the definition of early AMD, and its differentiation from features of normal aging, a distinction that remains poorly understood.

Histopathologically, the hallmark of early neovascular AMD is accumulation of extracellular drusen and basal laminar deposit (abnormal material located between the plasma membrane and basal lamina of the retinal pigment epithelium) and basal linear deposit (material located between the basal lamina of the retinal pigment epithelium and the inner collageneous zone of Bruch's membrane). The end stage of AMD is characterized by a complete degeneration of the neurosensory retina and of the underlying retinal pigment epithelium in the macular area. Advanced stages of AMD can be subdivided into geographic atrophy and exudative AMD. Geographic atrophy is characterized by progressive atrophy of the retinal pigment epithelium. In exudative AMD the key phenomenon is the occurrence of choroidal neovascularisation (CNV). Eyes with CNV have varying degrees of reduced visual acuity, depending on location, size, type and age of the neovascular lesion. The development of choroidal neovascular membranes can be considered a late complication in the natural course of the disease possibly due to tissue disruption (Bruch's membrane) and decompensation of the underlying longstanding processes of AMD.

Many pathophysiological aspects as well as vascular and environmental risk factors are associated with a progression of the disease, but little is known about the etiology of AMD itself as well as about the underlying processes of complications like the occurrence of CNV. Family, twin, segregation, and case-control studies suggest an involvement of genetic factors in the etiology of AMD. The extent of heritability, number of genes involved, and mechanisms underlying phenotypic heterogeneity, however, are unknown. The search for genes and markers related to AMD faces challenges—onset is late in life, and there is usually only one generation available for studies. The parents of patients are often deceased, and the children are too young to manifest the disease. Generally, the heredity of late-onset diseases has been difficult to estimate because of the uncertainties of the diagnosis in previous generations and the inability to diagnose AMD among the children of an affected individual. Even in the absence of the ambiguities in the diagnosis of AMD in previous generations, the late onset of the condition itself, natural death rates, and small family sizes result in underestimation of genetic forms of AMD, and in overestimation of rates of sporadic disease. Moreover, the phenotypic variability is considerable, and it is conceivable that the currently used diagnostic entity of AMD in fact represents a spectrum of underlying conditions with various genetic and environmental factors involved.

There remains a strong need for improved methods of diagnosing or prognosticating AMD or a susceptibility to AMD in subjects, as well as for evaluating and developing new methods of treatment. It is an object of the invention to identify inherited risk factors that suggest an increased risk in developing AMD or predicting the onset of more aggressive forms of the disease.

### SUMMARY

Peripheral retinal drusen and reticular pigment changes have been observed and described in subjects with and without AMD (Lewis, H. et al., Ophthalmol., 92:1485-1495, 1985). Standardized clinical exam forms were designed, therefore, to ascertain these peripheral retinal findings in genetic and epidemiologic studies of AMD. Photography of seven standard fields was included in the study protocols to expand the documentation of phenotype to the equatorial and mid-peripheral fundus. Preliminary analyses of data revealed an association between these peripheral retinal phenotypes and a family history of AMD.

Several genetic variants have been associated with various forms of age-related macular degeneration (AMD) including CFHY402H, CFHrs1410996, LOC387715A69S gene region, complement factor 2 (CF2), complement factor B (CFB), and complement factor 3 (C3) (references). The expanded analysis described herein identified the association between these known genetic variants and presence of non-macular drusen and pigment irregularities, thereby identifying, for example, another source of genetic susceptibility.

In one embodiment, the present invention is directed to a method for diagnosing a peripheral retinal phenotype or a susceptibility to a peripheral retinal phenotype comprising detecting one or more genetic markers associated with peripheral retinal phenotype. In a particular embodiment, the peripheral retinal phenotype is the presence of a certain pattern of peripheral retinal pigment called reticular pigment change or the presence of peripheral retinal drusen. In a particular embodiment, the one or more genetic markers are associated with CFH. In a particular embodiment, the one or more genetic markers comprise CFHY402H (SEQ ID NO:1 or CFHrs1410996 (SEQ ID NO:6). In a particular embodiment, detection of a the risk allele at CFHY402H or CFHrs1410996 is indicative of a peripheral retinal phenotype or susceptibility to a peripheral retinal phenotype. In a particular embodiment, the peripheral retinal phenotype is associated with age related macular degeneration.

One embodiment is directed to a method for identifying a carrier for peripheral retinal phenotype, comprising genotyping a subject wherein determining the subject is heterozygous for at least one risk allele or homozygous for two risk alleles indicates the subject is a carrier for a peripheral retinal phenotype. In a particular embodiment, the risk allele is CFHY402H or the C allele of CFHrs1410996. In a particular embodiment, the peripheral retinal phenotype is associated with age related macular degeneration.

One embodiment is directed to a kit for detecting a risk allele for a peripheral retinal phenotype, comprising a reagent for determining the allele at a polymorphic site associated with a peripheral retinal phenotype. In a particular embodiment, the polymorphic site is that of SEQ ID NO:1 or SEQ ID NO:6.

### BRIEF DESCRIPTION OF THE DRAWINGS

FIG. 1 is a plot showing sensitivities and specificities for a variety of risk score cutpoints and ROC curves for prediction of advanced age-related macular degeneration among younger and older age groups.

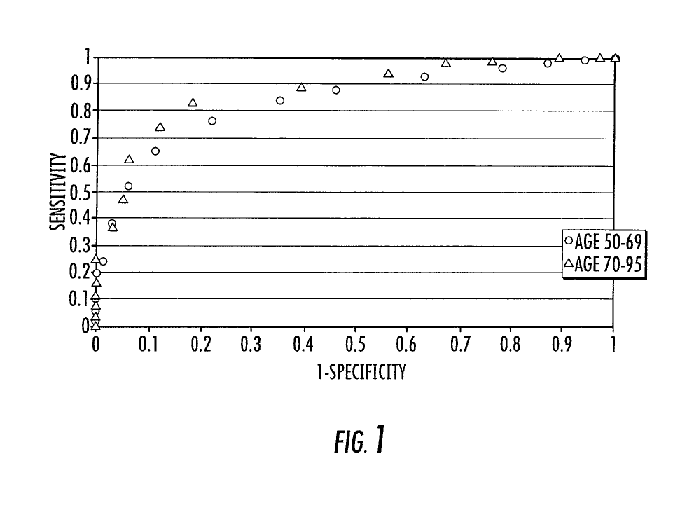

FIG. 2 shows plotted histograms for advanced age-related macular degeneration risk scores for cases and controls among the original sample (above) and replication sample (below) based on all genetic variants as well as demographic and environmental variables.

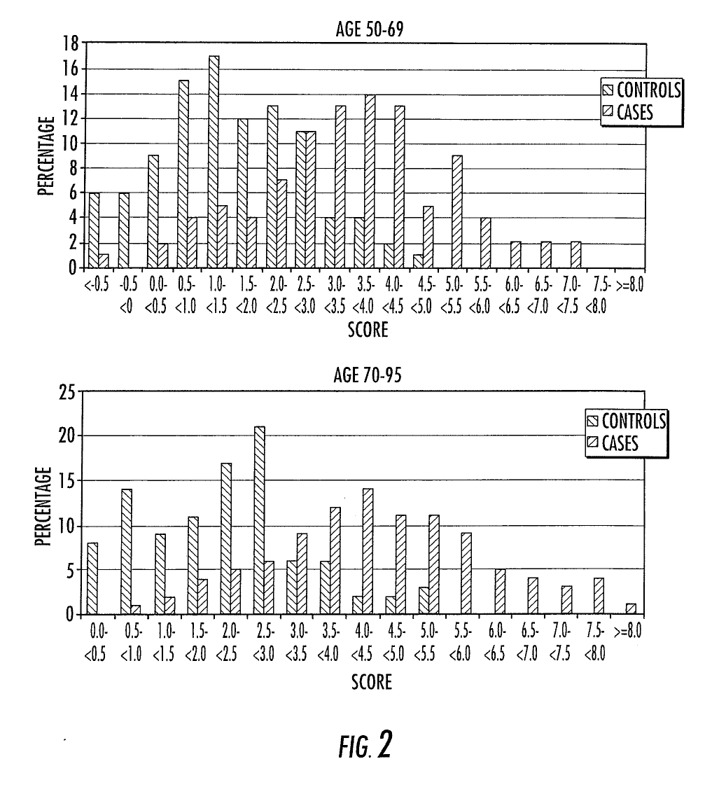

FIG. 3 are sequences showing alleles at polymorphic sites: rs2230199 (SEQ ID NO:1), rs1061170 (SEQ ID NO:2), rs10490924 (SEQ ID NO:3), rs9332739 (SEQ ID NO:4), rs641153 (SEQ ID NO:5), rs1410996 (SEQ ID NO:6) and rs2230203 (SEQ ID NO:7).

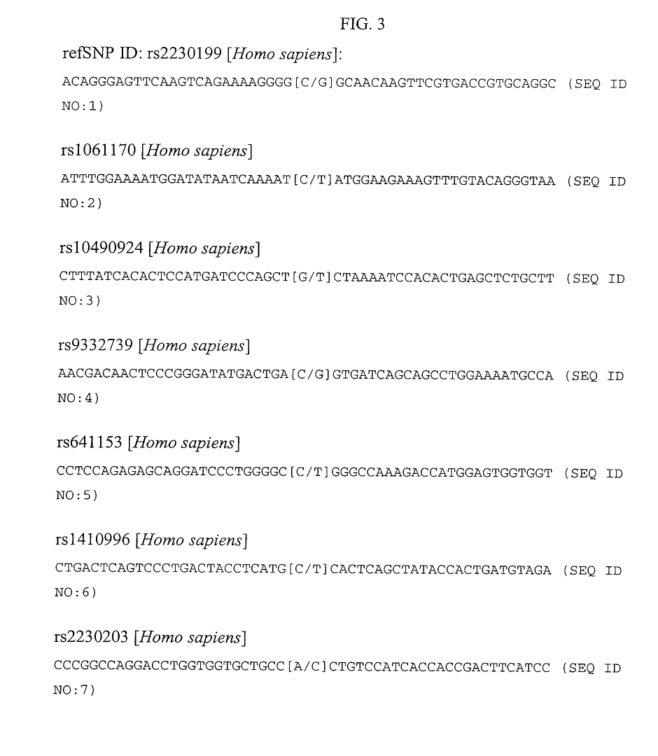

### DETAILED DESCRIPTION

The present invention is based on the unexpected discovery of genotypes associated with peripheral retinal drusen and reticular pigment changes, and genotypes associated with age-related macular degeneration (AMD). Genotypes were determined for six known variants associated with AMD. Associations between genotype and peripheral phenotypes for 2125 family members and twins were assessed using generalized estimating equations (GEE), which accounts for correlation between eyes.

In particular, complement factor H (“CFH”) genotype associations are described. Disclosed herein, for example, is the discovery of the association of peripheral retinal drusen and reticular pigment changes with CFHY402H and CFH rs1410996 genotypes in family and twin studies of AMD. Also described herein is the association of peripheral retinal phenotypes, e.g., peripheral retinal drusen and reticular pigment changes, with the diagnosis of AMD. These markers in the peripheral retina can be used to assess the genetics susceptibility for AMD in a subject.

Also described herein is the unexpected discovery that there is a strong association between peripheral retinal reticular pigment and CFH genotypes for individuals who have no or only minimal signs of AMD. This association is useful for identifying individuals who are carriers for the disease-associated alleles. The presence of peripheral drusen and reticular pigment was found to be associated with AMD (p<0.001). Both peripheral retinal phenotypes were also associated with AMD related genotypes. For CFHY402H, the p for trend was <0.001 for increase in peripheral drusen with each additional risk (C) allele, compared with the TT genotype (peripheral drusen present in 7% of TT, 10% of CT, and 18% of CC genotypes). Similar results were seen for CFHrs1410996 with p for trend=0.008 for increase in drusen with each additional risk allele (C), compared with the TT genotype (drusen present in 5% of TT, 10% of CT, and 14% of CC genotypes). Peripheral reticular pigment changes were also related to CFHY402H in both eyes, with p for trend <0.001 for increase in pigment with each risk allele (reticular pigment present in 18% of TT, 23% of CT, and 33% of CC genotypes). For CFHrs1410996, a similar association was seen (p for trend=0.010). Similar associations were not seen for the LOC387715 A69S gene region, C2 or CFB. For C3, there was a slight increase in presence of drusen and pigment with the GG risk genotype compared with the CC genotype, although this difference was not statistically significant. Even among individuals with no or minimal signs of AMD (grades 1 and 2), peripheral reticular pigment was associated with over a two-fold increased risk of having the AMD homozygous genotype for both CFH variants.

Peripheral retinal drusen and reticular pigment changes, therefore, are associated with CFHY402H and CFHrs1410996 genotypes. Clinically these peripheral findings are indicative of a genetic susceptibility even in non-AMD or low risk eyes. These analyses quantify for the first time the following: 1) the association between peripheral retinal drusen and peripheral reticular pigment and presence of AMD, 2) the association of these peripheral phenotypes with CFHrs1410996; 3) the relationship between peripheral retinal drusen and CFHY402H; and 4) the association between these peripheral retinal phenotypes and CFH genotypes among individuals without a diagnosis of AMD or with minimal signs of maculopathy.

As used herein, “gene” is a term used to describe a genetic element that gives rise to expression products (e.g., pre-mRNA, mRNA and polypeptides). A gene includes regulatory elements and sequences that otherwise appear to have only structural features, e.g., introns and untranslated regions.

The genetic markers are particular “alleles” at “polymorphic sites” associated with particular complement factors, e.g., CFH. A nucleotide position at which more than one nucleotide can be present in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). If at a particular chromosomal location, for example, one member of a population has an adenine and another member of the population has a thymine at the same genomic position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

A genetic marker is “associated” with a genetic element or phenotypic trait, for example, if the marker is co-present with the genetic element or phenotypic trait at a frequency that is higher than would be predicted by random assortment of alleles (based on the allele frequencies of the particular population). Association also indicates physical association, e.g., proximity in the genome or presence in a haplotype block, of a marker and a genetic element.

A reference sequence is typically referred to for a particular genetic element, e.g., a gene. Alleles that differ from the reference are referred to as “variant” alleles. The reference sequence, often chosen as the most frequently occurring allele or as the allele conferring a typical phenotype, is referred to as the “wild-type” allele.

Some variant alleles can include changes that affect a polypeptide, e.g., the polypeptide encoded by a complement pathway gene. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence.

Alternatively, a polymorphism associated with a peripheral retinal phenotype can be a synonymous change in one or more nucleotides (i.e., a change that does not result in a change to a codon of a complement pathway gene). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein are associated with peripheral retinal phenotypes (which are in turn associated with AMD and/or a susceptibility to AMD). Detection of the presence or absence of the haplotypes herein, therefore is indicative of peripheral retinal phenotypes, and, by extension, AMD or a susceptibility to AMD. The haplotypes described herein are a combination of genetic markers, e.g., SNPs and microsatellites. Detecting haplotypes, therefore, can be accomplished by methods known in the art for detecting sequences at polymorphic sites.

The haplotypes and markers disclosed herein are in “linkage disequilibrium” (LD) with preferred complement pathway genes, e.g., CFH, and likewise, peripheral retinal phenotypes, AMD and complement-associated phenotypes. “Linkage” refers to a higher than expected statistical association of genotypes and/or phenotypes with each other. LD refers to a non-random assortment of two genetic elements. If a particular genetic element (e.g., an allele at a polymorphic site), for example, occurs in a population at a frequency of 0.25 and another occurs at a frequency of 0.25, then the predicted occurrence of a person's having both elements is 0.125, assuming a random distribution of the elements. If, however, it is discovered that the two elements occur together at a frequency higher than 0.125, then the elements are said to be in LD since they tend to be inherited together at a higher frequency than what their independent allele frequencies would predict. Roughly speaking, LD is generally correlated with the frequency of recombination events between the two elements. Allele frequencies can be determined in a population, for example, by genotyping individuals in a population and determining the occurrence of each allele in the population. For populations of diploid individuals, e.g., human populations, individuals will typically have two alleles for each genetic element (e.g., a marker or gene).

The invention is also directed to markers identified in a “haplotype block” or “LD block”. These blocks are defined either by their physical proximity to a genetic element, e.g., a complement pathway gene, or by their “genetic distance” from the element. Other blocks would be apparent to one of skill in the art as genetic regions in LD with the preferred complement pathway gene, e.g., CFH. Markers and haplotypes identified in these blocks, because of their association with peripheral retinal phenotype(s) and the complement pathway, are encompassed by the invention. One of skill in the art will appreciate regions of chromosomes that recombine infrequently and regions of chromosomes that are “hotspots”, e.g., exhibiting frequent recombination events, are descriptive of LD blocks. Regions of infrequent recombination events bounded by hotspots will form a block that will be maintained during cell division. Thus, identification of a marker associated with a phenotype, wherein the marker is contained within an LD block, identifies the block as associated with the phenotype. Any marker identified within the block can therefore be used to indicate the phenotype.

Additional markers that are in LD with the markers of the invention or haplotypes are referred to herein as “surrogate” markers. Such a surrogate is a marker for another marker or another surrogate marker. Surrogate markers are themselves markers and are indicative of the presence of another marker, which is in turn indicative of either another marker or an associated phenotype.

### Identification of Complement Pathway Markers

SNPs associated with complement pathway genes were identified and assessed for their association with AMD (see Example 1). Tag SNPs were selected from across C3 and C5, including SNP rs2230199 in C3, which was reported to have a p=2.8×10⁻⁵ in single marker tests available on the NIH dbGAP database in a genome-wide association of 400 AMD cases and 200 controls. Genotyping was performed as part of experiments using the Illumina GoldenGate assay and Sequenom iPLEX system as previously described. The study population consisted of 2,172 unrelated Caucasian individuals 60 years of age or older diagnosed based on ocular examination and fundus photography (1,238 cases of both dry and neovascular (wet) advanced AMD and 934 controls).

A single SNP in C3 (rs2230199; SEQ ID NO:1) exhibited significant association to AMD, with p<10⁻¹² and minor allele frequency of 0.21 in controls and 0.31 in cases. This SNP creates a non-synonymous coding change (Arg102Gly) in the second exon of C3. No other SNPs typed in C3 showed individually statistically significant association. In addition to testing all individual genotyped SNPs, multi-marker haplotype tests were used to evaluate association at untyped SNPs present on HapMap but no additional associations were found. Association at these SNPs and haplotypes were tested further, conditioning on the genotype at rs2230199, and no significant associations were observed. Tests were also conducted to detect any difference in association between the neovascular and geographic atrophy forms of AMD. No statistically significant differences were observed. No SNPs in C5 exhibited significant association to AMD. The role of epistasis between rs2230199 and five variants was also evaluated. Two variants at CFH (1061170—SEQ ID NO:2 and 10490924—SEQ ID NO:3), two variants at the CFB/C2 locus (9332739—SEQ ID NO:4 and 641153—SEQ ID NO:5), and one at the LOC387715/HTRA1 locus (1410996—SEQ ID NO:6) were established as unequivocally associated to AMD risk in this cohort. Using logistic regression, no statistically significant interaction terms were observed between any pair of these SNPs, the two Factor B rare protective SNPs as a category or the three haplotypes formed by the two different CFH SNPs. While weak interactions cannot be excluded, this result suggests that despite targeting the same pathway, these variants largely confer risk in an independent, log-additive fashion.

Given the independent action of this new variant, rs2230199 it was added to the multi-locus risk model. Since the individual and combined effects of the AMD associated variants are additive, the overall proportion of population variance in risk (assuming a prevalence of late-stage AMD in this age group to be 5%) explained by this locus is roughly 2% (assuming an underlying normal distribution N(0,1) of risk across the population). For comparison, a comparable estimate of the effects of variation at CFH, LOC387715/HTRA1 and CFB are 16%, 10% and 2.5% respectively—indicating that the individual effects of these four identified genetic factors alone explain an impressive 30% of the population variation in risk for a late-onset complex disorder with known environmental covariates. Given the frequencies and penetrances of these alleles, these independent effects when combined create genuine predictive value for late-stage AMD in the population from which these cases and controls were drawn. While in this age group the prevalence of late-stage AMD is roughly 5%, variation at these four genes can identify 20% of the population that have less than 1% risk of disease, and at the opposite end identify 1% of the population with >50% risk. Indeed in this latter category, 154 cases (out of 1238) were identified compared to only 9 controls (out of 934).

HapMap Phase II reveals few proxies for rs2230199, with only 2 SNPs correlated with r²>0.4. The first, rs2230203 (SEQ ID NO:7), is a synonymous exonic polymorphism 7.6 kb downstream, correlated with r²=0.75. The other, is 5.9 kb upstream of rs2230199 outside of the gene, also correlated with r²=0.75. The small number of proxies together with the low level of linkage disequilibrium in the region suggest that the causal allele lies within a region spanning less than 14 kb.

This associated Arg102Gly variant (SEQ ID NO:1) has been established as the molecular basis of the two common allotypes of C3: C3F (fast) and C3S (slow), so named due to a difference in electrophoretic motility. The C3F variant has been previously reported as associated to other immune-mediated conditions such as IgA nephropathy and glomerular nephritis. The variant has also been reported to influence the long term success of renal transplants, where C3S homozygote recipients had much better graft survival and function when receiving a donor kidney with a C3F allotype than a matched homozygote C3S donor. More generally, deficiencies in both C3 and CFH have been associated to the immune-mediated renal damage in membranoproliferative glomerulonephritis (MPGN), and the AMD-associated Y402H variant has also been shown to be significantly associated with MPGN underscoring a deep connection in the etiology of these two disorders. The discovery of an additional association between variation in the complement system and AMD should serve to more precisely focus functional experiments and therapeutic development on the specific activity of the alternate pathway of the complement cascade.

### Diagnostic Gene Array

Polynucleotide arrays provide a high throughput technique that can assay a large number of polynucleotide sequences in a single sample. This technology can be used, for example, as a diagnostic tool to diagnose or assess the risk for developing a peripheral retinal phenotype. Peripheral retinal phenotypes can be associated, for example, with a risk potential of developing AMD. Polynucleotide arrays (for example, DNA or RNA arrays), are known in the art for use as diagnostic or screening tools. Such arrays include regions of usually different sequence polynucleotides arranged in a predetermined configuration on a substrate, at defined x and y coordinates. These regions (sometimes referenced as “features”) are positioned at respective locations (“addresses”) on a substrate. The arrays, when exposed to a sample, exhibit an observed binding pattern. This binding pattern can be detected upon interrogating the array. All polynucleotide targets (for example, DNA) in the sample can be labeled, for example, with a suitable label (e.g., a fluorescent compound) that allows for the detection of specific sample-array interactions. The observed binding pattern is indicative of the presence and/or concentration of one or more polynucleotide components of the sample.

Arrays can be fabricated by depositing previously obtained biopolymers onto a substrate, or by in situ synthesis methods. The substrate can be any supporting material to which polynucleotide probes can be attached, including but not limited to glass, nitrocellulose, silicon, and nylon. Polynucleotides can be bound to the substrate by either covalent bonds or by non-specific interactions, such as hydrophobic interactions. The in situ fabrication methods include those described in U.S. Pat. No. 5,449,754 for synthesizing peptide arrays, and in U.S. Pat. No. 6,180,351 and WO 98/41531 and the references cited therein for synthesizing polynucleotide arrays. Further details of fabricating biopolymer arrays are described in U.S. Pat. No. 6,242,266; U.S. Pat. No. 6,232,072; U.S. Pat. No. 6,180,351; U.S. Pat. No. 6,171,797; EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Other techniques for fabricating biopolymer arrays include known light directed synthesis techniques. Commercially available polynucleotide arrays, such as Affymetrix GeneChip™, can also be used. Use of the GeneChip™, to detect gene expression is described (Lockhart, D. et al., Nat. Biotech., 14:1675-1680, 1996; Chee, M. et al., Science, 274:610-614, 1996; Hacia, J. et al., Nat. Genet., 14:441-447, 1996; and Kozal, M. et al., Nat. Med., 2:753-759, 1996). Other types of arrays are known in the art, and are sufficient for developing an AMD diagnostic array of the present invention.

To create the arrays, single-stranded polynucleotide probes, for example, can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25 or 30 or more contiguous nucleotides selected from the nucleotide sequences shown in SEQ ID NO:1-7, e.g., SEQ ID NO:1 and SEQ ID NO:6. In array fabrication, the probes formed at each feature are usually expensive. Additionally, sample quantities available for testing are usually also very small and it is therefore desirable to simultaneously test the same sample against a large number of different probes on an array. These conditions make it desirable to produce arrays with large numbers of very small (for example, in the range of tens or one or two hundred microns), closely spaced features (for example many thousands of features).

Samples can be assayed directly for the presence or absence of one or more markers associated with a peripheral retinal phenotype. Samples can also be processed, for example, to isolate nucleic acids or to amplify specific nucleic acids. Tissue samples from a patient suspected of having or being at risk for developing a peripheral retinal phenotype, for example, can be treated to isolate single-stranded polynucleotides, for example by heating or by chemical denaturation, as is known in the art. The single-stranded polynucleotides in a tissue sample can be labeled and hybridized to the polynucleotide probes on the array. Detectable labels that can be used include but are not limited to radiolabels, biotinylated labels, fluorophors, and chemiluminescent labels. Double-stranded polynucleotides, comprising the labeled sample polynucleotides bound to polynucleotide probes, can be detected once the unbound portion of the sample is washed away. Detection can be visual or with computer assistance. Preferably, after the array has been exposed to a sample, the array is read with a reading apparatus (such as an array “scanner”) that detects the signals (such as a fluorescence pattern) from the array features. Such a reader would have a very fine resolution (for example, in the range of five to twenty microns) for a array having closely spaced features.

The signal image resulting from reading the array can be digitally processed to evaluate which regions of read data belong to a given feature as well as to calculate the total signal strength associated with each of the features. The foregoing steps, separately or collectively, are referred to as “feature extraction” (U.S. Pat. No. 7,206,438, for example, describes an apparatus and method of enhancing feature extraction, e.g., processing one or more detected signal images each acquired from a field of view of an array reader). Using any of the feature extraction techniques so described, detection of hybridization of a patient derived polynucleotide sample with one of the peripheral retinal phenotypic markers on the array given as SEQ ID NO:1-7 and markers associated with CFH identifies that patient as having a genetic risk factor for a peripheral retinal phenotype.

Also encompassed by the disclosure herein is a system for compiling and processing patient data, and presenting a risk profile for developing a peripheral retinal phenotype. A computer-aided medical data exchange system, for example, can be used. The system can be designed to provide high-quality medical care to a patient by facilitating the management of data available to care providers. The care providers include, for example, physicians, surgeons, nurses, clinicians, various specialists, and so forth. It should be noted, however, that while general reference is made to a clinician in the present context, the care providers may also include clerical staff, insurance companies, teachers and students, and so forth. The system provides an interface, which allows the clinicians to exchange data with a data processing system. The data processing system can be linked to an integrated knowledge base and a database. The system and the database draw upon data from a range of data resources. The database may be software-based and can include data access tools for drawing information from various resources, or coordinating or translating the access of such information. In general, the database unifies raw data into a useable faun.

The integrated knowledge base is intended to include one or more repositories of medical-related data. The data itself may relate to patient-specific characteristics as well as to non-patient specific information, as for classes of persons, machines, systems and so forth. Examples of patient-specific clinical data include patient medical histories, patient serum and cellular antioxidant levels, and the identification of past or current environmental, lifestyle and other factors that predispose a patient to develop AMD. These include but are not limited to various risk factors such as obesity, smoking, vitamin and dietary supplement intake, use of alcohol or drugs, poor diet and a sedentary lifestyle.

Use of the present system involves a clinician obtaining a patient sample, and evaluation of the presence of a genetic marker in that patient indicating a predisposition for a peripheral retinal phenotype, such as SEQ ID NO:1-7 (e.g., SEQ ID NO:1 and SEQ ID NO:6 and other CFH-associated markers). The clinician or their assistant also obtains appropriate clinical and non-clinical patient information, and inputs it into the system. The system then compiles and processes the data, and provides output information that includes a risk profile for the patient, of developing AMD. Particular illustrations of this process will depend on the specific information collected and the specific operations of the system, which are believed to be routine given the teachings provided herein.

Described herein are certain allelic markers, e.g., polynucleotide sequences, that have been correlated to AMD, compositions based on these markers, methods for using these markers, and kits and systems for practicing the methods using these markers. These markers are useful as diagnostics for identifying patients who have AMD, are at risk for developing AMD or who have a susceptibility to developing AMD. The markers described herein can be used alone or in conjunction with other diagnostic methods, e.g., methods using other markers or environmental risk factors.

### EXEMPLIFICATION

### Example 1

Several candidate genes have screened negatively for association with AMD (Haddad, S. et al., Surv. Ophthalmol., 50:306-363, 2006). The list includes TIMP3 (Tissue inhibitor of metalloproteinases-3), IMPG2, the gene encoding the retinal interphotoreceptor matrix (IPM) proteoglycan IPM 200, VMD2 (the bestrophin gene), ELOVL4 (elongation of very long chain fatty acids), RDS (peripherin), EFEMP1 (EGF-containing fibulin-like extracellular matrix), BMD (bestrophin). One gene has been shown to have variations in the coding regions in patients with AMD, namely, GPR75 (a G protein coupled receptor gene). Others have shown a possible association with the disease in at least one study; PON1 the (paraoxonase gene), SOD2 (manganese superoxide dismutase, APOE (apolipoprotein E) for which the ε4 allele has been found to be associated with the disease in some studies and not in others; and CST3 (cystatin C) for which one study has suggested an increased susceptibility for ARMD in CST3 B/B homozygotes. There are conflicting reports regarding the role of the ABCR (ABCA4) gene with regard to AMD. Allikmets and colleagues first reported an association with the disease.

Genetic variants associated with AMD have been identified. There is also an association to a region containing several tightly linked genes on chromosome 10 (LOC387715, HTRA1) although the function of those genes and variants is not fully understood. Using the database described herein, a previously unrecognized common, non-coding variant in CFH and other complement factor genes w was identified that substantially increases the influence of this locus on AMD, and strongly replicated the associations of four other published common alleles in three genes (p values ranging from 10-12 to 10-70), including the first confirmation of the BF/C2 locus.

Complement Pathway is involved in AMD: Genetic variants and environment play a role in AMD development and pathogenesis. Therefore, it is desirable to take both into account when determining an individual's risk. To date, the Y402H variant of complement factor H (CFH) and the rs1410996 variant contribute to AMD along with other CFH variants, and the confer about a 3-fold increased risk in patients with the homozygous condition. The Y402H single nucleotide polymorphism (SNP) is within the CFH binding site for heparin and C-reactive protein (CRP). Altered binding to these sites can lead to loss of function; e.g., decreased ability to bind to targets and/or interact with CRP, thereby giving rise to excessive complement activation. Because the initiation of complement activation can occur on cell surfaces as well as in the fluid phase, the activation of complement is one of the earliest events that can be detected.

When classical pathway activation occurs through the binding and activation of C1 to antibodies, C4 is cleaved, producing C4a and C4b. C4a is released locally and is circulated. It can be detected by a commercially available ELISA kits (e.g., Pharmingen OPT-EIA) in ng/mL quantities. A similar event occurs when the lectin pathway is activated through binding of mannose binding lectin (MBL) to a carbohydrate-covered bacterial surface and the mannan-binding lectin-associated serine protease (MASP) enzymes cleave C4. C4a thus serves as a marker for activation of both the classical and lectin pathways. Many charged surfaces on microbes or other particulates including aggregates of multiple classes of immunoglobulins have been shown to activate the alternative complement pathway. The first split product released in this pathway is Bb from the cleavage of factor B. Bb can be measured in plasma by a commercial ELISA kit (e.g., Quidel) in μg/mL quantities. As complement pathways can interact with one another, measuring components of each pathway may be important for diagnosis or prediction of complement-associated disease, e.g., AMD.

If activation by any of the pathways continues, C3 is the next major protein to produce measurable fragments. C3 is initially split into two pieces: C3a is a small fragment that has anaphylatoxin activity, interacting through a specific C3a receptor found on many cell types, and C3b is a large fragment that has the property of binding covalently to nearby surfaces or molecules through an active thioester bond. The latter is produced by a conformational change in the molecule when the C3 convertase cleaves it. This covalent attachment leads to permanent deposits of C3b (or its subsequent cleavage fragments) on surfaces in the vicinity of complement activation. These deposits and subsequent cleavage fragments interact with C3 receptors (CR1, CR2, CR3, CR4) that are found on many cell types. This leads to immune adherence and provides a transport mechanism for the clearance of immune complexes, bacteria, viruses or whatever the C3b has become attached to. C5a and C5b-9 (membrane attack complex (MAC)) are markers of the terminal activation pathway as well.

CFH dampens the alternative pathway by three actions: 1) it prevents binding of factor B to C3; b) it binds to C3bBb (the alternative pathway C3 convertase), displacing the Bb enzymatic subunit; and 3) it provides cofactor activity for factor I (CFI), which can then cleave C3b, producing the inactive form, iC3b. Some iC3b is in the fluid-phase in concentrations normally below 30 μg/mL in plasma, with low variability. When elevated, it may provide an indirect indication that CFH is functioning to inactivate C3b. Inhibition of CFH with antibody reduces the cleavage of C3b to iC3b as measured by Western blot. To determine the function of CFH in inactivating C3b, it would be desirable to measure C3b and iC3b. C3b assays, however, show substantial variability. C3, which reflects certain disease states, is therefore measured, and the ratio of iC3b/C3 is analyzed as another possible indicator of AMD risk.

Factor B provides the enzymatic subunit, Bb, of the C3 convertase, contributing to the amplification loop of the alternative pathway and formation of C5 convertase. Whereas CFH dampens the alternative pathway, properdin stabilizes C3 and C5 convertases of the alternative pathway, thus serving to promote formation of the MAC instead of inactivation of C3b. Whereas variants of CFH increase the risk of AMD, variations in the genes encoding factor B were found to reduce the risk of AMD. Both factors B and C3 are important in the development of laser-induced choroidal neovascularization in mouse models.

In addition to genetic considerations, environmental factors play a role in AMD risk and may affect complement levels. Smoking is an independent risk factor for AMD and has been reported to activate complement and to increase factor B levels. Smokers have been reported to have reduced CFH levels. Plasma levels of CFH are reported to vary widely in the general population (110-615 μg/mL) and the measurement of CFH may not differentiate normal from variant CFH. To identify at-risk patients, therefore, other possible biomarkers associated with AMD are measured—biomarkers that may also be affected by environmental factors strongly associated with increased risk of AMD. Based on the pathways, it would be anticipated that iC3b (or iC3b/C3) would be most elevated in non-smokers with the CFH Y402H TT genotype and with low BMI (anticipated to have stage 1), and undetectable in CC smokers with high BMI and with advanced AMD. For CC smokers with stage 1, it would be expected that factor B levels would be lower than in those with advanced AMD (with the possible caveat of patients with protective variants of factor B). Bb, a fragment of factor B produced by activation of the alternative pathway, is a reliable marker of alternative pathway activation. Ratios of Bb to B are informative with respect to the activation rate and extent of the alternative pathway, and analysis of these factors in conjunction with C3 measures provides insight into the processes ongoing in the inflammatory lesions.

Genetic approach to AMD: AMD falls into the category of complex, late-onset diseases similar to type II diabetes, Alzheimer's disease, cardiovascular disease, hypertension, etc. where the genetic contributions do not necessarily manifest with straightforward Mendelian inheritance. Instead, it is presumed that these and other complex diseases are the result of complex interaction between environmental factors and susceptibility of multiple alleles of multiple genes and that these factors only cause disease when, in combination, a threshold of susceptibility is reached. Two major hypotheses are commonly explored to search for these genetic risk factors—the “common disease/common variant hypothesis” (e.g., as suggested by the association of the APOE4 allele with Alzheimer's disease) and the hypothesis that rarer, more penetrant variants at multiple genes may explain the genetic component of multifactorial disease. While there is not general agreement, and limited empirical data, to suggest which hypothesis will bear more fruit in any individual disease, it seems most likely that complex diseases with involvement of many genes may quite naturally have contributions from both common and rare variation.

To detect common, low-penetrance variation, the association study is the design of choice—as made evident by both theoretical considerations and a proven track-record of detecting common genetic variants for multifactorial disease. Common variation has been conclusively determined to play a substantial role in the heritability of AMD. Previous efforts, however, have focused almost exclusively on polymorphisms that are already known to result in changes in the coding and regulatory regions of genes. A limited knowledge of the genome, limited ability to recognize many forms of potentially functional variation from sequence context alone, and lack of true understanding of causal pathways, has therefore limited the ability to apply these techniques (which remain quite costly and unproven). These hurdles have been overcome and recent results indicate that successful identification and replication of low-penetrance alleles can be convincingly achieved. Plasma biomarkers in the complement system are associated with AMD and AMD progression, and these associations differ according to genotype, controlling for environmental factors.

Baseline plasma levels of the complement factors are measured in patients who are genotyped and phenotyped for AMD to determine if these markers predict risk of AMD given environmental risk factors. The study population includes: 1) Discordant sibling pairs (from families and DZ twins) with one sibling grade 3b, 4, and 5 and one sibling with grade 1 (N=100 pairs, with 200 siblings), and 2) Progressors among the siblings with transition from grades 1-4 to grades 3b, 4, and 5 or grade 4 to 5 over time (total sample 620 of whom 214 have progressed). There will be additional progressors over time and the total sample expected for this aim is approximately 1000 subjects. All subjects have stored plasma samples that have never been thawed, and were collected in a manner that can be used for these lab analyses. Plasma data can be coupled with risk factor data as described above, including smoking, body mass index (BMI) and serum high-sensitivity CRP from a different aliquot of blood drawn on the same day as the proposed plasma complement assays (for the discordant pairs). Serum CRP and plasma complement factors (from aliquots drawn on the same day at baseline) can also be measured for subjects in the progression aspect of the study for the prospective analyses.

Complement assays: CFH, factor B, factor I, C3 and C5 levels are measured primarily with radial immunodiffusion, using polyclonal antisera specific for the components, according to the procedures followed by the Complement Laboratory at NJC. Split products C3a, iC3b, C5a and C4a, along with the terminal complement complex (SC5b-9), are measured by ELISA using kits produced by Pharmingen BD or Quidel. Ratios (iC3b:C3 and C3a:C3) can be calculated with these data. The normal ranges for these components are given in Table 1.

TABLE 1

| | Normal Range | |
|---|---|---|
| Component | (mean ± 2 standard deviations) | |
| Factor H | 160-412 | μg/mL |
| Factor I | 29-58 | μg/mL |
| Factor B | 127.6-278.5 | μg/mL |
| C3 | 66-162 | mg/dL |
| C5 | 55-113 | μg/mL |
| C4 | 11-39 | mg/dL |
| C3a | 98-857 | ng/mL |
| iC3b | 0-30.9 | μg/mL |
| Bb | 0-0.83 | μg/mL |
| SC5b-9 | 0-179 | ng/mL |
| C4a | 101-745 | ng/mL |

In the clinical laboratory, anything outside of three standard deviations is considered abnormal. Given that some of the patients may have low native components (C3, FB and C4), the ratio of the levels to the split products are predicted to be more useful than absolute amounts. Comparison of the results from the disease cohorts with the controls is extremely useful for further studies in terms of identifying the appropriate biomarkers for AMD patients. All complement split products are evaluated in specimens that have been collected in EDTA tubes, processed to obtain the EDTA-plasma rapidly after blood collection, and stored frozen in liquid nitrogen freezers. Each specimen is tested for all proteins on the first thaw, since repeated freeze-thaw cycles can produce false positive results.

Methods—CFH, factor I, factor B, C5: Radial immunodiffusion is performed by preparing 1% agarose gels containing an appropriate amount of specific antibody for the component to be measured. Wells are cut in the gel and filled with a measured amount of each test serum or plasma, control serum or plasma, and a series of at least three standards with known concentration of the component measured. After incubation of the filled gels for 72 hours at 4° C., the diameter of the precipitin ring formed by combination of the antibody with its antigen (the component being tested) is measured and the area of the precipitin ring is calculated. Using the areas of the rings formed by the standards, the concentrations of the component present in the test samples are calculated by linear regression.

C3a, C4a: ELISA method using OptEIA kits from Pharmingen-BD (San Diego). iC3b, Bb, SC5b-9: these markers are measured using kits from Quidel (San Diego, Calif.). Three controls are run with each set of test samples, and the specimens are all tested in duplicate.

C-reactive protein (CRP) binds to CFH at the CCP7 where the Y402H CFH polymorphism exists. Serum CRP was found to be elevated in patients with AMD compared to controls. CRP may also increase the risk of AMD in patients carrying at least one allele of the CFH variant. While not being bound by a particular theory, it has been proposed that CFH binds CRP and counter-arrests alternative pathway activation induced by damaged tissue.

Analyses: For the case-control comparison, conditional logistic regression was used to determine the likelihood of having advanced AMD given levels of the various complement factors and CRP values within categories of genotype, while assessing and adjusting for pack year history of smoking, BMI and cardiovascular disease. Effect modification between complement factors versus CRP and complement factors versus genotype is also determined. Risk factor data is available within the existing database and analyzed. Additional analyses are performed to assess associations between genotype and complement factors using the general linear model. For progression, Cox regression analyses is applied to assess whether complement levels are associated with AMD progression, controlling for genotype, smoking, BMI, CRP, etc. Interactions and effect modification are assessed to determine if complement factors are more or less related to AMD within certain genotypes, or whether these associations vary depending on smoking status, level of BMI, etc. Power for the discordant pair analyses is adequate to detect an effect size (i.e., mean difference between groups/sd)=0.40 with 80% power based on a comparison of 100 cases and 100 controls. Power is even larger for the progression study where there are 214 progressors out of 620 subjects. Regarding multiple testing, the different complement factors tend to be highly correlated and a Bonferroni type correction would be inappropriate.

### Example 2

### Methods

Study Population: Participants in the family genetic study of AMD (N=1619) and the U.S. twin study (N=506) included herein were examined according to standardized examination and photography protocols and AMD grading systems designed for these studies. Baseline examinations were conducted. Follow-up ocular records were obtained and follow-up examinations and photography were conducted.

Phenotypes—Grade of AMD: AMD grade was determined based on clinical and photographic data using the Clinical Age-Related Maculopathy Grading System (CARMS), which has a 5 step scale with grade 1=no AMD or only a few hard drusen, grade 2a=several hard drusen or a few intermediate size drusen, grade 2b=pigment irregularities, grade 2c=both hard drusen and pigment irregularities, grade 3a=large soft drusen or several intermediate size drusen, grade 3b=drusenoid retinal pigment epithelial detachment (RPED), grade 4=geographic atrophy-both central and non-central, grade 5=neovascular disease or serous RPED. Visual acuity was not a criteria for assigning AMD grade. The most recent grade in each eye was used in the analyses.

Peripheral Retinal Phenotypes: Standardized clinical examination forms designed for the family and twin studies, as well as other AMD studies, incorporate questions about the presence of peripheral drusen and peripheral reticular pigment changes. Example photographs for peripheral reticular pigment are also provided. Furthermore, photographs of seven fields are obtained as part of the study protocol to document equatorial and peripheral retinal drusen and pigment changes. All photographs are reviewed and the presence or absence of drusen in all locations are recorded.

Genotyping: DNA samples were obtained from whole blood and stored in a blood repository. The following six common single nucleotide polymorphisms (SNPs) associated with AMD were evaluated: 1) Complement Factor H (CFH)Y402H (rs1061170) in exon 9 of the CFH gene on chromosome 1q31, a change 1277T>C, resulting in a substitution of histidine for tyrosine at codon 402 of the CFH protein, 2) CFH rs1410996, which is an independently associated SNP variant within intron 14 of CFH, 3) LOC387715 A69S (rs10490924 in the LOC387715/HTRA1 region of chromosome 10), a non-synonymous coding SNP variant in exon 1 of LOC387715, resulting in a substitution of the amino acid serine for alanine at codon 69, 4) Complement Factor 2 or C2 E318D (rs9332739), the non-synonymous coding SNP variant in exon 7 of C2 resulting in the amino acid glutamic acid changing to aspartic acid at codon 318, 5) Complement Factor B or CFB R32Q (rs641153), the non-synonymous coding SNP variant in exon 2 of CFB resulting in the amino acid glutamine changing to arginine at codon 32, 6) Complement Factor 3 or C3 R102G (rs2230199), the non-synonymous coding SNP variant in exon 3 of C3 resulting in the amino acid glycine to arginine at codon 102. For the genetic variant on chromosome 10, LOC387715A69S, it remains uncertain whether the gene HTRA1 adjacent to it may in fact be the AMD-susceptibility gene on 10q26 31-33 references; but the relevant SNPs in these 2 genes have been reported to be nearly perfectly correlated. Thus, while the other SNP is a promising candidate variant, rs10490924 used in this study can be considered a surrogate marker for the causal variant that resides in this region. Genotyping was performed using primer mass extension and MALDI-TOF MS analysis by the MassEXTEND methodology of Sequenom (San Diego, Calif.).

Statistical Analyses: Distributions of baseline demographic, genotypic and ocular characteristics for the family, twin and combined datasets were calculated and displayed in Tables 2 and 3. For Table 4, the relationship between peripheral drusen and AMD grade as a categorical variable was assessed using PROC GENMOD of SAS. Similar analyses were run for peripheral reticular pigment. Table 5 was created based on GEE analyses using PROC GENMOD of SAS with a logit link regressing the logit of the probability of peripheral drusen on AMD grade and genotype as categories. In addition, tests for trend were calculated for the number of non-wild type alleles for genes with more than two genotypes using a similar approach. For all analyses the eye was the unit of analyses and the correlation between eyes was taken into account.

Table 2 displays the demographic and genetic characteristics of the study population. Only Caucasian participants were included in these analyses. The mean age for the total population is 76.9 (±8.6) years, with 55% males and 45% females. Ocular characteristics were similar for OD and OS as shown in Table 3. Twenty-eight percent were grade 1 (no AMD) and about 37% of eyes had advanced AMD (grades 4 or 5). Peripheral drusen were present in 11%-12% of eyes, and peripheral reticular pigment was present in 24%-25% of eyes.

TABLE 2
Baseline Demographic Genetic Characteristics of Participants

| | Family | | Twin | | Total | |
|---|---|---|---|---|---|---|
| | N = 1619 | | N = 506 | | N = 2125 | |
| Mean Age | 76.6 +/− 9.4 | | 77.8 +/− 5.1 | | 76.9 +/− 8.6 | |
| (+/− SD) | | | | | | |
| Gender | | | | | | |
| M (%) | 665 | (41%) | 100% | | 1170 | (55%) |
| F (%) | 955 | (59%) | | | 955 | (45%) |
| Genotype | | | | | | |
| CFH Y420H: rs1061170 | | | | | | |
| TT | 330 | (21) | 157 | (32) | 487 | (24) |
| CT | 733 | (47) | 221 | (45) | 954 | (46) |
| CC | 508 | (32) | 112 | (23) | 620 | (30) |
| CFH: rs 1410996 | | | | | | |
| TT | 114 | (7) | 64 | (13) | 174 | (9) |
| CT | 569 | (36) | 196 | (42) | 765 | (38) |
| CC | 880 | (56) | 213 | (45) | 1093 | (54) |
| LOC987715: | | | | | | |
| rs10490924 (A69S) | | | | | | |
| GG | 591 | (38) | 263 | (54) | 854 | (42) |
| GT | 675 | (44) | 182 | (38) | 857 | (42) |
| TT | 280 | (18) | 40 | (8) | 320 | (16) |
| C2: rs9332739 (E318D) | | | | | | |
| GG | 1447 | (93) | 449 | (93) | 1896 | (93) |
| CG/CC | 117 | (7) | 35 | (7) | 152 | (7) |
| CFB: rs641153 (R32Q) | | | | | | |
| CC | 1351 | (87) | 429 | (88) | 1780 | (87) |
| CT/TT | 204 | (13) | 57 | (12) | 261 | (13) |
| C3: rs2330199 (R102G) | | | | | | |
| CC | 712 | (49) | 218 | (48) | 930 | (49) |
| CG | 631 | (49) | 190 | (42) | 821 | (43) |
| GG | 115 | (8) | 45 | (10) | 160 | (8) |

TABLE 3
Ocular Characteristics of Participants

| | | | Twin | | | |
|---|---|---|---|---|---|---|
| | | | Total | | | |
| | OD | OS | OD | OS | OD | OS |
| AMD Grade | N (%) | | N(%) | | N(%) | |
| 1 | 361 (23) | 367 (23) | 217 (43) | 218 (43) | 578 (28) | 585 (28) |
| 2 | 229 (14) | 229 (14) | 122 (24) | 121 (24) | 351 (17) | 352 (17) |
| 3 | 293 (19) | 309 (19) | 89 (18) | 76 (15) | 382 (18) | 385 (18) |
| 4 | 240 (15) | 254 (16) | 30 (6) | 32 (6) | 270 (13) | 286 (14) |
| 5 | 458 (29) | 428 (27) | 34 (9) | 55 (11) | 502 (24) | 483 (23) |
| Peripheral Drusen (%) | 150 (12) | 155 (13) | 25 (9) | 23 (8) | 175 (11) | 178 (12) |
| Reticular Pigment (%) | 313 (25) | 305 (25) | 62 (22) | 64 (23) | 375 (25) | 369 (24) |

Table 4 displays the association between AMD grade and peripheral retinal drusen and reticular pigment. The presence of peripheral drusen increased from 7% for grades 1 and 2, to between 13% and 17% for grades 3-5 (p for trend <0.001). The presence of reticular pigment increased from 14% to 15% for grades 1 and 2 to 25% for grade 3, and 34% to 35% for grades 4 and 5 (p for trend <0.001).

TABLE 4
Association Between Peripheral Retinal Drusen and Reticular Pigment and AMD Grade

| | AMD Grade | | | | | |
|---|---|---|---|---|---|---|
| | 1 | 2 | 3 | 4 | 5 | P (Trend) |
| | No. (%) | | | | | |
| Peripheral | 54/821 (7) | 31/437 (7) | 83/500 (17) | 52/406 (13) | 126/808 (16) | |
| Drusen | | | | | | |
| OR (CI) | 1.0 | 1.1 (0.7-1.7) | 2.8 (2.0-4.1) | 2.1 (1.4-3.1) | 2.6 (1.9-3.7) | |
| P Value | | 0.73 | <0.001 | <0.001 | <0.001 | <0.001 |
| Peripheral | 112/813 (14) | 67/433 (15) | 125/502 (25) | 141/403 (35) | 278/808 (34) | |
| Pigment | | | | | | |
| OR (CI) | 1.0 | 1.1 (0.8-1.6) | 2.1 (1.6-2.8) | 3.4 (2.5-4.5) | 3.3 (2.6-4.2) | |
| P Value | 0.42 | <0.001 | <0.001 | <0.001 | <0.001 | <0.001 |

Table 5 reveals the distribution of peripheral drusen and pigment according to genotype overall. The presence of peripheral drusen was associated with CFHY402H in both eyes, with p for trend <0.001 for increase in drusen with each additional risk allele (C) allele, compared with the TT genotype (drusen present in 7% of TT, 10% of CT, and 18% of CC genotypes). Similar results were seen for CFHrs1410996 with a p for trend=0.008 for increase in drusen with each additional risk allele (C), compared with the TT genotype (drusen present in 5% of TT, 10% of CT, and 14% of CC genotypes). Peripheral reticular pigment changes were also related to CFHY402H in both eyes, with p for trend <0.001 for increase in pigment with each risk allele (reticular pigment present in 18% of TT, 23% of CT, and 33% of CC genotypes). For CFHrs1410996, a similar association was observed for peripheral pigment (p for trend=0.010), which was present in 18% of TT, 21% of CT, and 28% of CC genotypes. Similar associations were not observed for the LOC387715 A69S gene region, C2 or CFB. For LOC387715, the trend was actually in the “protective direction” for both peripheral drusen and pigment with each additional AMD risk allele. For C3, there was a slight increase in percent of drusen and pigment with the GG risk genotype compared with the CC genotype, but this difference was not statistically significant.

TABLE 5
Distribution of Peripheral Retinal Drusen and Reticular Pigment According to Genotype

| | Peripheral Drusen | | | Reticular Pigment | | |
|---|---|---|---|---|---|---|
| Genotype | N (%) | | OR (CI) | N (%) | | OR (CI) |
| CFHY402H: rs1061170 (Y420H) | | | | | | |
| TT | 42/639 | (7) | 1.0 | 113/637 | (18) | 1.0 |
| CT | 140/1395 | (10) | 1.4 (1.0-2.0) | 315/1397 | (23) | 1.2 (0.9-1.6) |
| CC | 168/920 | (18) | 2.6 (1.8-3.7) | 297/909 | (33) | 1.8 (1.4-2.3) |
| P (trend) | <0.001 | | | <0.001 | | |
| CFH: rs1410996 | | | | | | |
| TT | 10/219 | (5) | 1.0 | 40/217 | (18) | 1.0 |
| CT | 111/1084 | (10) | 2.1 (1.1-4.1) | 226/1083 | (21) | 1.1 (0.7-1.6) |
| CC | 224/1639 | (14) | 2.5 (1.3-4.8) | 464/1629 | (28) | 1.4 (0.9-2.0) |
| P (trend) | 0.008 | | | 0.010 | | |
| LOC387715: RS10490924 (A69S) | | | | | | |
| GG | 149/1132 | (13) | 1.0 | 297/1125 | (26) | 1.0 |
| GT | 128/1248 | (10) | 0.6 (0.5-0.8) | 305/1242 | (25) | 0.8 (0.6-0.9) |
| TT | 61/506 | (12) | 0.7 (0.5-0.9) | 101/506 | (20) | 0.5 (0.3-0.6) |
| P (trend) | 0.002 | | | <0.001 | | |
| C2: rs9332739 (E318D) | | | | | | |
| GG | 315/2686 | (12) | 1.0 | 669/2675 | (25) | 1.0 |
| GG/CC | 27/229 | (12) | 1.2 (0.8-1.8) | 50/229 | (22) | 1.0 (0.7-1.4) |
| P (trend) | 0.43 | | | 0.97 | | |
| CFB: rs641153 (R32Q) | | | | | | |
| CC | 291/2512 | (12) | 1.0 | 620/2498 | (25) | 1.0 |
| CC/TT | 42/387 | (11) | 1.0 (0.7-1.5) | 94/389 | (24) | 1.1 (0.8-1.4) |
| P (trend) | 0.82 | | | 0.66 | | |
| C3: rs2230199 (R102G) | | | | | | |
| CC | 151/1289 | (12) | 1.0 | 298/1290 | (23) | 1.0 |
| CG | 121/1203 | (10) | 0.8 (0.6-1.0) | 296/1192 | (25) | 1.1 (0.9-1.3) |
| GG | 34/228 | (15) | 1.2 (0.8-1.9) | 58/226 | (26) | 1.1 (0.8-1.6) |

| | | |
|---|---|---|
| P (trend) | 0.83 | 0.31 |

Table 6 shows the distribution of peripheral drusen and pigment according to genotype among individuals without AMD or with only minimal signs of maculopathy (grades 1 and 2 only). For both of the CFH genotypes, reticular pigment was significantly associated with increasing number of risk alleles (p<0.001 for CFHY402H, and p=0.018 for CFHrs1410996). There was a greater than two-fold increased risk for having peripheral drusen with the presence of one or two risk alleles for both genotypes. Similar associations were not seen for the other genotypes.

TABLE 6
Distribution of Peripheral Drusen and Pigment According to
Genotype for Grades 1 and 2

| | Peripheral Drusen | | | Reticular Pigment | | |
|---|---|---|---|---|---|---|
| Genotype | N (%) | | OR (CI) | N (%) | | OR(CI) |
| CFHY402H: | | | | | | |
| rs1061170 | | | | | | |
| (Y402H) | | | | | | |
| TT | 14/354 | (4) | 1.0 | 33/352 | (9) | 1.0 |
| CT | 46/564 | (8) | 2.2 (1.2-4.0) | 89/560 | (15) | 1.8 (1.2-2.8) |
| CC | 19/245 | (8) | 2.0 (1.0-4.1) | 48/241 | (20) | 2.4 (1.5-3.8) |
| P (trend) | 0.045 | | | <0.001 | | |
| CFH: | | | | | | |
| rs1410996 | | | | | | |
| TT | 4/140 | (3) | 1.0 | 11/138 | (8) | 1.0 |
| CT | 39/514 | (8) | 2.8 (1.0-7.9) | 75/508 | (15) | 2.0 (1.0-3.9) |
| CC | 33/496 | (7) | 2.4 (0.8-6.9) | 83/492 | (17) | 2.3 (1.2-4.5) |
| P (trend) | 0.34 | | | 0.018 | | |
| LOC387715: | | | | | | |
| rs10490924 | | | | | | |
| (A69S) | | | | | | |
| GG | | | 1.0 | | | 1.0 |
| GT | | | 0.5 (0.3-0.9) | | | 0.8 (0.6-1.2) |
| TT | | | 0.9 (0.4-2.0) | | | 0.3 (0.1-0.7) |
| P (trend) | 0.14 | | | 0.009 | | |
| C2: | | | | | | |
| rs9332739 | | | | | | |
| (E318D) | | | | | | |
| GG | 7/1011 | (7) | 1.0 | 149/1001 | (15) | 1.0 |
| CG/CC | 5/121 | (4) | 0.6 (0.2-1.4) | 17/121 | (14) | 1.0 (0.6-1.6) |
| P (trend) | 0.23 | | | 0.86 | | |
| CFB: | | | | | | |
| rs641153 | | | | | | |
| (R32Q) | | | | | | |
| CC | 63/950 | (7) | 1.0 | 139/940 | (15) | 1.0 |
| CT/TT | 15/191 | (8) | 1.2 (0.7-2.2) | 26/191 | (14) | 0.9 (0.6-1.5) |
| P (trend) | 0.52 | | | 0.73 | | |
| C3: | | | | | | |
| rs2230199 | | | | | | |
| (R102H) | | | | | | |
| CC | 40/535 | (7) | 1.0 | 76/533 | (14) | 1.0 |
| CG | 24/449 | (5) | 0.7 | 66/441 | (15) | 1.1 |
| GG | 6/79 | (8) | 1.0 | 8/79 | (10) | 0.6 |

| | | |
|---|---|---|
| P (trend) | 0.40 | 0.59 |

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. All references cited herein and throughout this specification are hereby incorporated herein by reference in their entirety.

What is claimed is:

1. A method for diagnosing a peripheral retinal phenotype or a susceptibility to a peripheral retinal phenotype comprising detecting one or more genetic markers associated with peripheral retinal phenotype.

2. The method of claim 1, wherein the peripheral retinal phenotype is reticular pigment change or the presence of peripheral retinal drusen.

3. The method of claim 1, wherein the one or more genetic markers are associated with CFH.

4. The method of claim 3, wherein the one or more genetic markers comprise CFHY402H (SEQ ID NO:1 or CFHrs1410996 (SEQ ID NO:6).

5. The method of claim 4, wherein detection of a the risk allele at CFHY402H or CFHrs1410996 is indicative of a peripheral retinal phenotype or susceptibility to a peripheral retinal phenotype.

6. The method of claim 1, wherein the peripheral retinal phenotype is associated with age related macular degeneration.

7. A method for identifying a carrier for peripheral retinal phenotype, comprising genotyping a subject wherein determining the subject is heterozygous for at least one risk allele or homozygous for two risk alleles indicates the subject is a carrier for a peripheral retinal phenotype.

8. The method of claim 7, wherein the risk allele is CFHY402H or the C allele of CFHrs1410996.

9. The method of claim 7, wherein the peripheral retinal phenotype is associated with age related macular degeneration.

10. A kit for detecting a risk allele for a peripheral retinal phenotype, comprising a reagent for determining the allele at a polymorphic site associated with a peripheral retinal phenotype.

11. The kit of claim 10, wherein the polymorphic site is that of SEQ ID NO:1 or SEQ ID NO:6.


SEQUENCE LISTING (7 sequences)

SEQ ID NO: 1 - DNA, 52 bp, Homo sapiens
acagggagttcaagtcagaaaaggggsgcaacaagttcgtgaccgtgcaggc

SEQ ID NO: 2 - DNA, 52 bp, Homo sapiens
atttggaaaatggatataatcaaaatyatggaagaaagtttgtacagggtaa

SEQ ID NO: 3 - DNA, 52 bp, Homo sapiens
ctttatcacactccatgatcccagctkctaaaatccacactgagctctgctt

SEQ ID NO: 4 - DNA, 52 bp, Homo sapiens
aacgacaactcccgggatatgactgasgtgatcagcagcctggaaaatgcca

SEQ ID NO: 5 - DNA, 52 bp, Homo sapiens
cctccagagagcaggatccctggggcygggccaaagaccatggagtggtggt

SEQ ID NO: 6 - DNA, 52 bp, Homo sapiens
ctgactcagtccctgactacctcatgycactcagctataccactgatgtaga

SEQ ID NO: 7 - DNA, 52 bp, Homo sapiens
cccggccaggacctggtggtgctgccmctgtccatcaccaccgacttcatcc